(19) United States
(12) Patent Application Publication
(10) Pub. No.: US 2025/0273337 A1
(43) Pub. Date: Aug. 28, 2025

(54) NUCLEIC ACID ERROR SUPPRESSION

(71) Applicant: Cornell University, Ithaca, NY (US)

(72) Inventors: Dan-Avi Landau, New York, NY (US); Alexandre Pellan Cheng, New York, NY (US)

(21) Appl. No.: 19/190,355

(22) Filed: Apr. 25, 2025

Related U.S. Application Data
(63) Continuation of application No. PCT/US2023/035877, filed on Oct. 25, 2023.
(60) Provisional application No. 63/380,915, filed on Oct. 25, 2022.

(51) Int. Cl. G16H 50/20; C12N 15/10; C12Q 1/6806; C12Q 1/6809; C12Q 1/6848; C12Q 1/6855; C12Q 1/686; C12Q 1/6869; C12Q 1/6886; G06N 20/00; G16B 20/20; G16B 30/10; G16B 40/20

(52) U.S. Cl. CPC G16H 50/20; C12N 15/1093; C12Q 1/6806; C12Q 1/6809; C12Q 1/6848; C12Q 1/6855; C12Q 1/686; C12Q 1/6869; C12Q 1/6886; G06N 20/00; G16B 20/20; G16B 30/10; G16B 40/20

(57) ABSTRACT

Nucleic acid error suppression is provided. In various embodiments, DNA is extracted from a collection of plasma samples. A sequence library with duplex adapters is prepared by ligating a duplex adapter having a Unique Molecule Identifier (UMI) to an end of each of a plurality of strands of the extracted DNA and amplifying the extracted DNA with a first polymerase chain reaction (PCR). A subset of the whole genome library is selected and amplified with a second PCR to increase an amount of PCR duplicates. A plurality of duplex reads is sequenced from the amplified subset aligned to a host genome and denoised based on said alignment. A variant presence is detected in at least one of the plurality of duplex reads. A signature of the variant is determined, which is compared to a collection of disease-specific variant signatures. A disease type is determined based on the comparison.

### RELATED APPLICATION(S)

This application is a continuation of International Application No. PCT/US2023/035877, filed Oct. 25, 2023, which claims the benefit of priority to U.S. Provisional Application No. 63/380,915, filed Oct. 25, 2022, each of which is hereby incorporated by reference in its entirety.

### TECHNICAL FIELD

The invention relates generally to the field of medical diagnostics. In particular, embodiments of the disclosure relate to methods and systems for reducing sequencing error rates in cancer detection and other fields requiring low error sequencing.

### BACKGROUND

Monitoring circulating cell-free DNA (cfDNA) has been shown to be a promising clinical tool for non-invasive cancer detection. While analysis of cancer-specific epigenetic markers, such as DNA methylation and histone modifications, has been applied to cfDNA for detection of various cancers, mutation-based approaches using direct genomic sequencing of somatic variants found in circulating tumor DNA (ctDNA) afford more specificity and clinically-actionable information. As such, ctDNA genome sequencing is preferable for clinical applications, particularly in cases where there is low burden of disease, such as early cancer screening, detection of minimal residual disease (MRD) after treatment or surgery and relapse monitoring of emergent resistant mutations for guided therapy. In these scenarios, tumor fraction is low, such that robust detection requires methods with exquisite sensitivity.

Prevailing methods of ctDNA detection use targeted sequencing protocols, which increase the number of genomes sequenced at a targeted location. However, high throughput targeted sequencing rapidly exhausts available genomes for sequencing (1,000-10,000 genome equivalents (GEs) per mL of plasma), which sets a design-based ceiling on ctDNA detection, where further increasing sequencing depth at a targeted site affords no advantage after the limited number of GEs has already been sequenced. Alternatively, to overcome these limitations, whole genome sequencing (WGS) approaches exploit breadth of coverage to supplant depth, eliminating the reliance on the detection of a single site to increase ctDNA characterization in low tumor fraction settings. For example, recent method M R Detect uses primary tumor mutational profiles to inform genome-wide tumor single nucleotide variant (SNV) detection in cfDNA, such that the available number of GEs no longer is the limiting factor for successful ctDNA detection.

The detection challenges presented by sparsity are significant, calling for broad, accurate and deep cfDNA sequencing. Thus, whole-genome, low-error, high-coverage methods are necessary for robust ctDNA analysis. However, the costs associated with these approaches are often prohibitive, particularly for clinical application. Although genome sequencing costs have rapidly dropped since the introduction of high-throughput next generation sequencing, more recently this decrease has stagnated. As such, sequencing cost is still a significant barrier to implementation of high-depth WGS for liquid biopsies, where in clinically important applications, tumor fractions are low (˜10-5) and shallow WGS is insufficient for ctDNA detection. To estimate the cost of implementing WGS for successful detection of ctDNA at such fractions, the probability of detecting a single mutation in a cfDNA sample can be modeled, given the number of GEs, the tumor fraction and sequencing depth¹⁷. It is estimated that a sequencing depth of over 100× is required for single mutation detection in tumor-derived DNA at this low fraction level, making the per-sample cost of WGS exceedingly high for application at scale with established technologies (˜2,000 USD per sample using an Illumina Novaseq S4 flow cell with v1.5 reagent costs).

Recently, a new low-cost, high-throughput sequencing method utilizing mostly natural sequencing-by-synthesis (mnSBS) has been developed by Ultima Genomics. The Ultima sequencing platform produces single-end reads at ˜10 billion reads per run for 1$/GB, thus substantially lowering sequencing costs compared with current platforms. This cost efficiency holds great promise for many genomics applications, and this approach has now been applied to Genome-In-A-Bottle and 1000 Genomes reference samples and adapted for single-cell RNA-seq studies. However, mnSBS/Ultima sequencing has not been harnessed for application to clinical cfDNA samples for ctDNA sequencing. In addition, notably, the error rate profiles of this new sequencing method have not been fully characterized, nor have they been rigorously compared with competing technologies. Importantly, for potential application to clinical disease monitoring of ctDNA, it is especially crucial to have accurate error rate estimates due to the high sensitivity of low-burden ctDNA detection.

Accordingly, there is a need for methods of reducing sequencing error rates in disease detection. The technical challenges imparted by the sparsity of ctDNA in low-burden disease settings may be overcome by increasing sequencing depth, accompanied by a low error rate. Unique molecular identifier (UMI) error suppression techniques or duplex sequencing, can increase accuracy in differentiating true somatic variant calls from errors introduced by sequencing, and may be combined with deep genomic sequencing to optimize successful detection of low-burden disease in a clinical setting.

### SUMMARY

Here, to investigate the utility of deep WGS for ctDNA detection, Ultima Genomics' mnSBS sequencing platform is used to sequence circulating cell-free DNA reads from plasma samples from healthy controls, cancer patients and patient-derived xenograft mouse models. In a first proof-of-principle study, it is shows that deep WGS (˜100×) with in silico error correction allows ctDNA detection within the part per million range. By leveraging the cost-effective and high-throughput nature of Ultima Genomics sequencing (109 reads per run at 1$/Gb), high coverage duplex-sequencing libraries of cell-free DNA can be produced, achieving error rates as low as 2.7×10-7. This allows accurate assessment of disease burden in post-treatment melanoma patients in the absence of any tumor information. Together, this demonstrates the utility of deep WGS in clinical samples for ctDNA detection.

According to certain aspects of the present disclosure, systems and methods are disclosed for detecting cancer with a lower sequencing error rate.

In an embodiment, a method comprises extracting DNA from a collection of plasma samples; preparing a whole genome library with duplex adapters, wherein the whole genome library is prepared by ligating a duplex adapter having a Unique Molecule Identifier (UMI) to an end of each of a plurality of strands of the extracted DNA and amplifying the extracted DNA with a first PCR; selecting a subset of the whole genome library; amplifying the subset with a second PCR to increase a number of PCR duplicates; sequencing a plurality of duplex reads from the amplified subset; aligning the plurality of duplex reads to a host genome and denoising the plurality of duplex reads based on said alignment; detecting the presence of a variant in at least one of the plurality of duplex reads; determining a signature of the variant; comparing the signature of the variant to a collection of disease-specific variant signatures; and determining a disease type based on the comparison.

### BRIEF DESCRIPTION OF THE DRAWINGS

The accompanying drawings, which are incorporated into and constitute a part of this specification, illustrate various exemplary embodiments and together with the description, serve to explain the principles of the disclosed embodiments.

FIG. 1 is a flowchart illustrating a method for detecting mutation signatures to determine a cancer status, according to techniques disclosed herein.

FIG. 2A is a series of graphs illustrating error rates and sequencing coverage for tumor fractions at or below 10⁻⁵, according to techniques disclosed herein.

FIG. 2B is a flowchart of a pre-analytical process to prepare a cfDNA library, according to techniques disclosed herein.

FIG. 2C is a graph illustrating sequencing depths for matched Illumina and Ultima datasets, according to techniques disclosed herein.

FIG. 2D is a comparison of normalized read coverage of a sequenced matched cfDNA sample, according to techniques disclosed herein.

FIG. 2E is a comparison of copy number-based variant (CNV) and single-nucleotide variant (SNV) tumor fractions, according to techniques disclosed herein.

FIG. 2F is a graph of an in silico mixing study, according to techniques disclosed herein.

FIG. 3A is a series of graphs illustrating duplex whole genome sequencing (WGS) on a mouse (left) and patient (right) sample, according to techniques disclosed herein.

FIG. 3B is a graphical comparison of variant allele frequencies calculated using unfiltered sequencing reads, according to techniques disclosed herein.

FIG. 3C is a graph comparing the model allele fraction of a patient with progressive disease in duplex corrected positions and copy-number based tumor fraction estimations, according to techniques disclosed herein.

FIG. 3D is a graph illustrating exemplary trinucleotide frequencies from a melanoma-associated UV signature.

FIG. 3E is a comparison of cosine similarities with either the SBS7 or the SBS1B mutation across conditions, according to techniques disclosed herein.

FIG. 3F is a graph illustrating a signature score and ctDNA detection of an in silico mixing study of metastatic melanoma samples, according to techniques disclosed herein.

FIG. 3G is a graph illustrating a series of signature scores of melanoma signature 7 in plasma cfDNA samples using duplex WGS, according to techniques disclosed herein.

FIG. 3H is a graph illustrating estimated tumor fraction of samples with elevated signature scores, according to techniques disclosed herein.

FIG. 4 is a series of graphs illustrating frequency of cfDNA fragment lengths in single-end Ultima sequencing datasets matched with paired-end Illumina sequencing, according to techniques disclosed herein.

FIG. 5A is a graph illustrating the UG specific blacklist, according to techniques disclosed herein.

FIG. 5B is an illustration of overlap between the UG blacklist and other low confidence regions, according to techniques disclosed herein.

FIG. 5C is a graph illustrating the overlap between melanoma tumor tissue SNVs and low confidence regions, according to techniques disclosed herein.

FIG. 6A is a heatmap of cosine similarities in cancer-free samples and high-burden ctDNA samples, according to techniques disclosed herein.

FIG. 6B is a boxplot of cosine similarities for three correction methods in the same cancer-free samples and high-burden ctDNA samples, according to techniques disclosed herein.

FIG. 7A is a graph illustrating a deconvolution of duplex-corrected mutations into representative mutational signatures.

FIG. 7B is a correlation plot between age at cancer diagnosis and number of clock-like mutations attributed to SBS1A and SBS1B, according to techniques disclosed herein.

FIG. 8 is a graph of a tumor-agnostic copy-number based tumor fraction estimation in cancer-free control samples and pre-surgery melanoma plasma, according to techniques disclosed herein.

FIG. 9A is a graph illustrating a homopolymer size between two PCR duplicates, according to techniques disclosed herein.

FIG. 9B is a graph illustrating a homopolymer size between a read and an aligned reference, according to techniques disclosed herein.

FIG. 9C is a graph illustrating frequency of homopolymer size across the human genome, according to techniques disclosed herein.

FIG. 9D is a graph illustrating indel calling accuracy by PCR duplicate family sizes, according to techniques disclosed herein.

FIG. 10 is a graph illustrating a single nucleotide variant analysis of matched Ultima and Illumina sequencing datasets, according to techniques disclosed herein.

FIG. 11A is a flow chart of a sequencing process providing predictable, error-robust motifs, according to techniques disclosed herein.

FIG. 11B isa graph of error rate by sequencing platform, according to techniques disclosed herein.

FIG. 12A is a graph of duplex WGS libraries from three starting inputs sequenced at 1-13× coverage, according to techniques disclosed herein.

FIG. 12B is a graph illustrating a duplication rate of the samples of FIG. 12A.

FIG. 12C is a graph of the effect of downsampling experiments, according to techniques disclosed herein.

FIG. 12D is a graph illustrating that duplex coverage is significantly higher at fixed coverage, according to techniques disclosed herein.

FIG. 12E is a graph illustrating a number of duplex variants found using fgbio versus a decision tree, according to techniques disclosed herein.

FIG. 13 is a graph illustrating mutational error rates in mouse PDX samples, according to techniques disclosed herein.

FIG. 14 is a bar graph illustrating a number of pre-surgery samples represented in validation experiments, according to techniques disclosed herein.

FIG. 15 is a graph illustrating detection of a chemotherapy mutational signature in plasma-free DNA, according to techniques disclosed herein.

FIG. 16A is a bar graph illustrating an apobec signature and measurement, according to techniques disclosed herein.

FIG. 16B is a bar graph illustrating an apobec signature and measurement, according to techniques disclosed herein.

FIG. 17 is a computing node according to embodiments of the present disclosure.

### DETAILED DESCRIPTION

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

Cell-free DNA (cfDNA) sequencing for low-burden cancer monitoring is limited by sparsity of circulating tumor DNA (ctDNA), the abundance of genomic material within a plasma sample, and pre-analytical error rates due to library preparation and sequencing errors. Sequencing costs have historically favored the development of deep targeted sequencing approaches for overcoming sparsity in ctDNA detection, but these techniques are limited by the abundance of cfDNA in samples, which imposes a ceiling on the maximal depth of coverage in targeted panels.

Whole genome sequencing (WGS) is an orthogonal approach to ctDNA-based cancer detection that can overcome the low abundance of cfDNA, supplanting breadth for depth by integrating signal across the entire tumoral mutation landscape. However, the higher cost of WGS limits practical depth of coverage and broad adoption.

Lower sequencing costs allows for enhanced ctDNA cancer monitoring via WGS. Emerging lower-cost WGS (Ultima Genomics, 1$/Gb) were applied to plasma samples at ˜120× coverage. Copy number and single nucleotide variation profiles are comparable between matched Ultima and Illumina datasets, however the deeper WGS coverage enables ctDNA detection at the parts per million range. These lower sequencing costs are further harnessed to implement duplex error-corrected sequencing at the scale of the entire genome, demonstrating a ˜3,000× decrease in errors in the plasma of patient-derived xenograft mouse models when compared to raw sequencing reads, and error rates as low as ˜10-7 in plasma samples from patients with metastatic melanoma. The highly de-noised plasma WGS is leveraged to undertake cancer monitoring in the more challenging context of low burden melanoma without matched tumor sequencing. In this context, duplex-corrected WGS allowed us to harness known mutational signature patterns for disease monitoring without matched tumors, paving the way for de novo cancer monitoring.

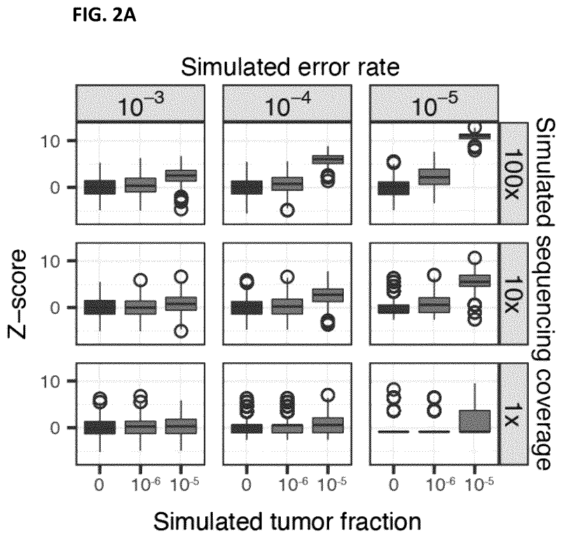

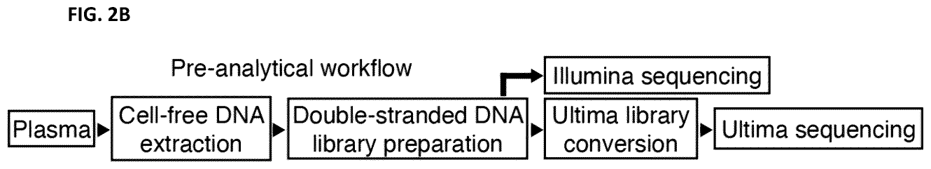

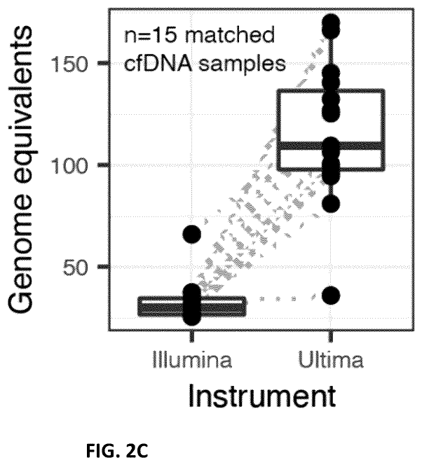

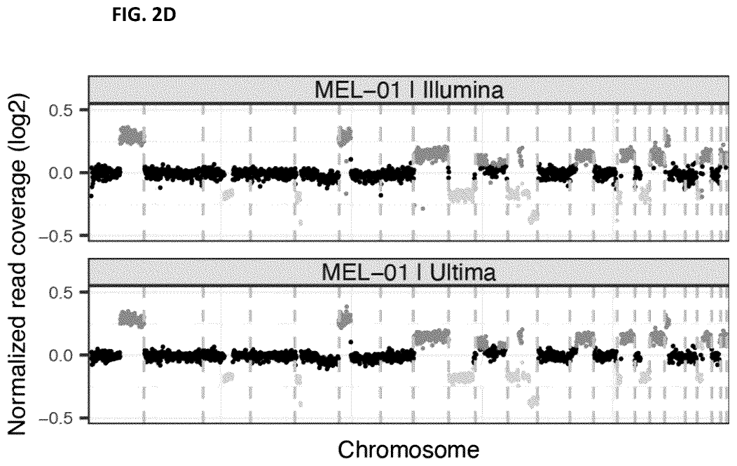

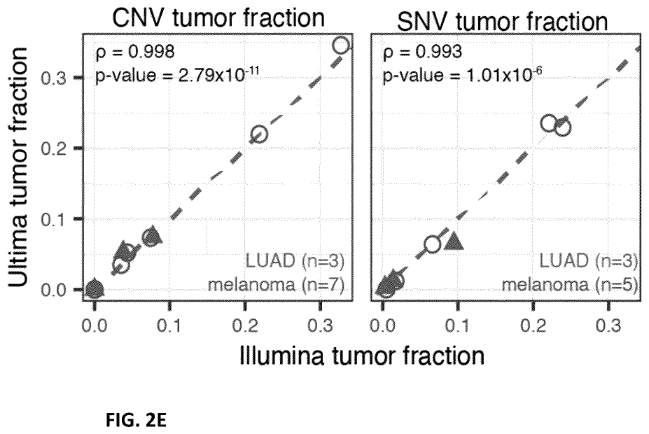

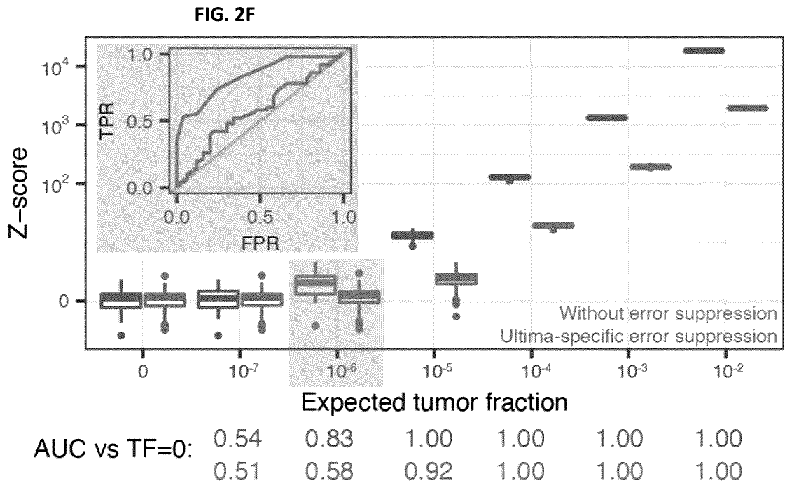

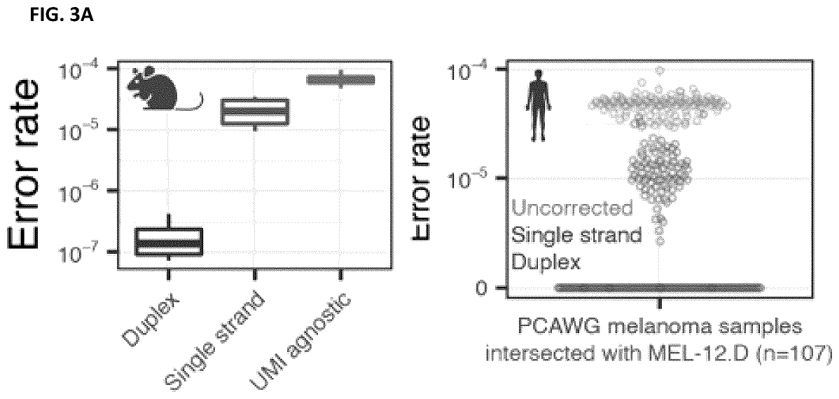

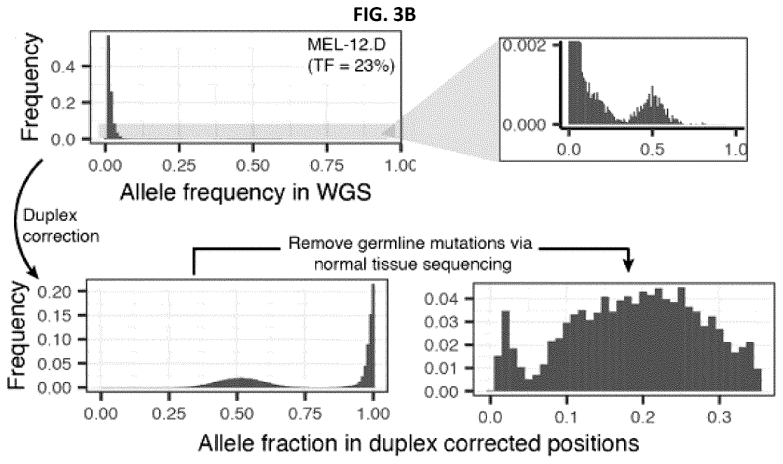

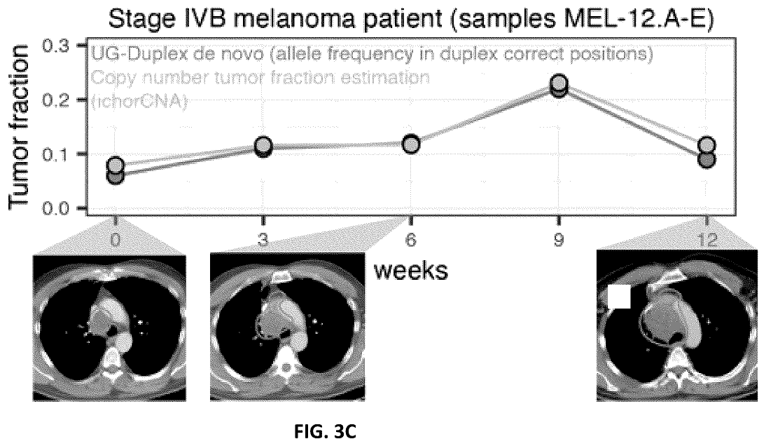

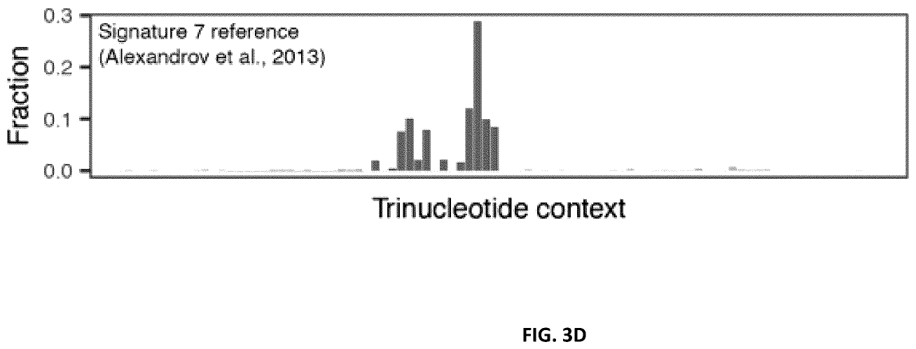

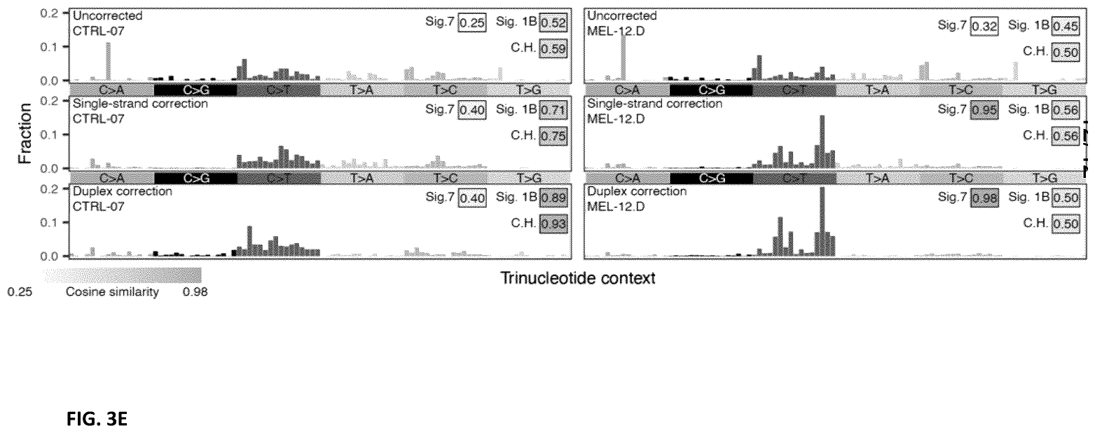

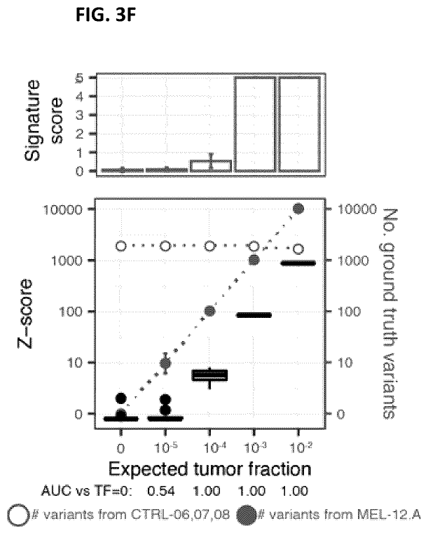

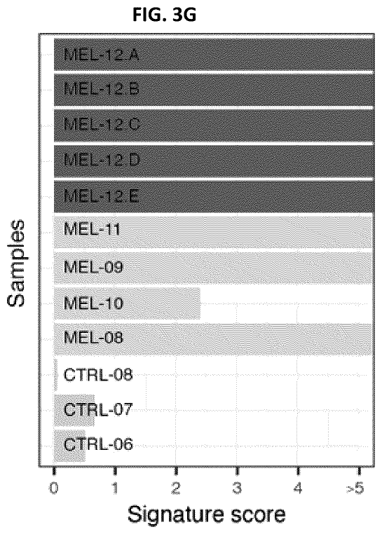

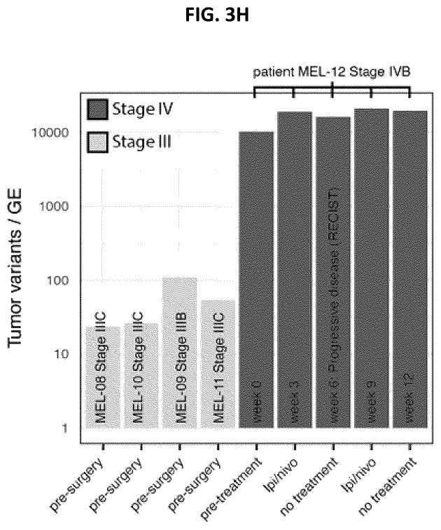

Deep WGS may be used for ctDNA detection with low-pass sequencing. Low-cost WGS (Ultima Genomics, $1/Gb) may be used to plasma samples at 120× coverage. Copy number and single nucleotide variation profiles were comparable between matched Ultima and Illumina datasets, however the deeper WGS coverage enabled ctDNA detection at the parts per million range. These lower sequencing costs were further harnessed to implement duplex error-corrected at the scale of the entire genome, demonstrating a ˜3000× decrease in errors in the plasma of patient-derived xenograft mouse models when compared to raw sequencing reads, and error rates as low as ˜10⁻⁷ in plasma samples from patients with metastatic melanoma. The highly de-noised plasma WGS was leveraged to undertake cancer monitoring in the more challenging context of low burden melanoma without matched tumor sequencing. In this context, duplex-corrected WGS allowed the harnessing of known mutational signature patterns for disease monitoring without matched tumors, paving the way for de novo cancer monitoring. Sequencing may be done using Ultima Genomics' sequencing platform to sequence 2.6×10⁹±1.4×10⁸ circulating cfDNA reads from 31 plasma samples (n=8 healthy controls; n=19 cancer patient samples; n=4 patient-derived xenograft mouse samples). Deep WGS (˜100×) with in silico error correction allows ctDNA detection within the part per million range over the sequenced genome. The cost-effective and ultra high-throughput nature of Ultima Genomics sequencing (10⁹ reads per run at 1$/Gb) can produce 4.15±3.23 coverage (range 1.02-11.55) duplex-sequencing libraries of cfDNA and achieve error rates as low as 2.7×10⁻⁷. Together, the results of sequencing and running the in silico error correction demonstrate the feasibility and utility of deep WGS in clinical samples for ctDNA detection. High-throughput short-read sequencing has revolutionized the liquid biopsy field, and sequencing costs have historically decreased at a rate faster than Moore's Law. However, the decrease in cost has stagnated over the past decade. The emergence of a high-throughput, low-cost sequencing platform would allow for deeper and broader sequencing of samples and patient populations, respectively. To work towards this goal, a comparative analysis of Ultima and Illumina short-read sequencing platforms was performed, demonstrating that these two approaches have comparable tumor-informed analysis capabilities for circulating tumor DNA detection (FIG. 2A-2F). Importantly, these tumor-informed approaches allow for ctDNA detection even at the part per million range, thereby making such analysis suitable for minimal residual disease detection. To further expand and demonstrate the utility of the technology, the more challenging problem of ctDNA detection in tumor-agnostic settings, where disease status or tumor origin is unknown, was addressed. For this important clinical context, whole-genome duplex correction was employed to achieve low error rates, allowing us to deconvolve the cell-free DNA mutational compendium into representative mutational signatures to detect ctDNA in the pre-operative setting, without matched tumor sequencing (FIGS. 3A-3H). Compared to commonly-used off-the-shelf panels, whole-genome analysis has the benefit of sequencing breadth, allowing for the detection of rare tumor-derived mutations that may not be present in targeted panels. One of ordinary skill in the art may envision that the methods can be harnessed for de novo cancer monitoring in low burden disease scenarios, providing a powerful tool for diagnosing cancer and detecting relapses at the earliest stages, leading to better patient outcomes overall. In addition to de novo cancer detection, the method can be used for cancer screening (e.g., screening for bladder cancer, melanoma, lung cancer, lynch syndrome cancer, BRCA syndrome cancers, based on APOBEC, UV, tobacco, MSI, and BRCA signatures, respectively).

It is noted that this method is not only useful for de novo detection (e.g. signatures) for tumor monitoring, but also for using tumor informed approaches. In this case, the duplex sequencing will decrease error and provide enhanced signal resolution for detection of a compendia of tumor-confirmed mutations. FIG. 2C illustrates the use of a tumor informed approach, which does not rely on signature analysis.

In an embodiment, this method is not only useful for monitoring, but also for non-invasive whole genome characterization of mutations in cancer (for example to identify actionable driver mutations or mutations that stratify patients to specific therapies). This is done again via reducing error of various sorts. FIG. 2B illustrates this characterization, which does not rely on signature analysis.

In an embodiment, this method enables non-invasive detection and discovery of driver mutations in somatic mosaicism. FIG. 2E shows detection of non-malignant mutation, specifically, detection of clonal hematopoiesis mutations.

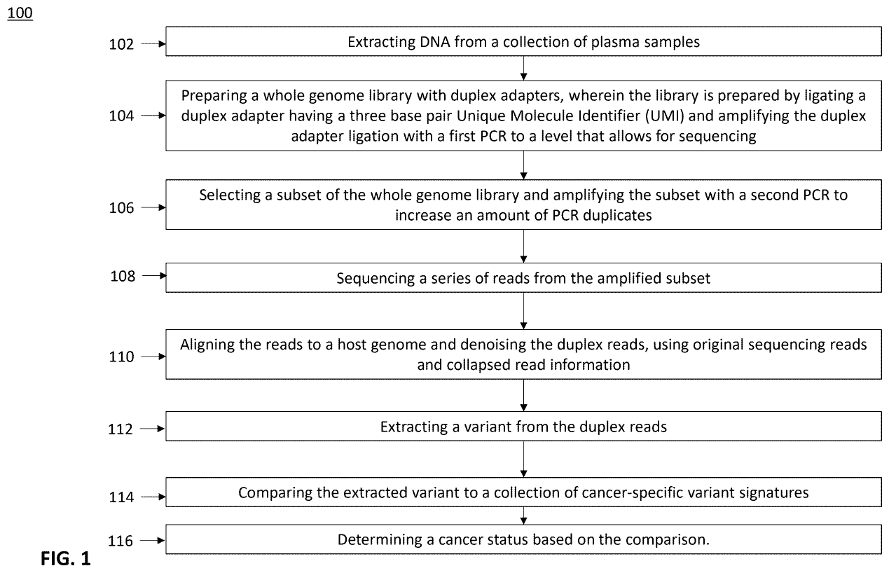

FIG. 1 is a flowchart illustrating an exemplary method for detecting variants in DNA using duplex sequencing and denoising, according to an exemplary embodiment of the present disclosure. For example, an exemplary method 100 (e.g., steps 102-118) may be performed, in part, by a processor automatically or in response to a request by a user.

According to one embodiment, the exemplary method 100 for detecting variants may include one or more of the following steps. In step 102 the method includes extracting DNA from a collection of plasma samples. Plasma can be from any human fluid, including urine, saliva, peritoneal fluid, cerebral spinal fluid, etc. The extracted DNA may be cfDNA or genomic DNA.

Genomic DNA can be extracted using the QiA amp DNA Mini Kit (Qiagen, cat #563034) and the QiA amp DNA blood Kit (Qiagen, cat #51104) for tissue and blood samples, respectively, and sheared to 450 bp (Covari). In an exemplary experiment, sequencing libraries were prepared on 1 μg of DNA using the TruSeq DNA PCR-Free Library Preparation Kit (Illumina), with one additional bead cleanup performed after end-repair and after adapter ligation. Extracted DNA was quantified using a Qubit 3.0 fluorometer and length analysis was performed using an Agilent Bioanalyzer or High Sensitivity Fragment Analyzer. 2×150 bp paired-end sequencing was performed on either a HiSeq X or NovaSeq v1.0 Illumina machine.

Cell-free DNA can be extracted from plasma using the Magbind cfDNA extraction kit (Omega Biotek, M 3298). Manufacturer recommendations for extraction were followed, but elution volume was increased to 35 uL and elution time was increased to 20 minutes on a thermomixer at 1,600 rpm (room temperature). Extracted cfDNA was quantified using a Qubit 3.0 fluorometer and length analysis was performed using an Agilent Bioanalyzer or High Sensitivity Fragment Analyzer.

In step 104, the method includes preparing a whole genome library with duplex adapters. In some embodiment, the library is prepared by ligating a duplex adapter having a three base pair Unique Molecule Identifier (UMI) and amplifying an amount of DNA with a first PCR to a level that allows for sequencing.

The duplex adapters contain a three base pair UMI that allows for tracing a top strand of the DNA to a bottom strand of the native DNA molecule. In rare mutation settings such as early detection and minimal residual disease, ctDNA content can fall below 1 in 10,000 concentrations. Therefore, in 1 mL of plasma containing 1,000-10,000 GEs, at most 1 circulating tumor read can be expected to overlap each somatic locus, which is lower than the per-base error rate of high-throughput sequencers (˜1 error per 1000 bases). To overcome this issue, deep-targeted sequencing approaches can use UMIs that are incorporated during library preparation for sequencing error correction. While strand-agnostic UMIs can help collapse sequencing errors, UMIs that link forward and reverse DNA strands (i.e. Duplex sequencing) can be used to collapse errors that arise on one strand (such as G>T transversions due to oxidative DNA damage) or during library preparation.

Extracted cfDNA libraries can be generated in a similar fashion as in Illumina whole genome sequencing, although the full-length adapters are replaced with stubby Y-adapters containing the three UMI bases (IDT Duplex Seq adapters (1080799)).

A first PCR amplification creates a set of PCR duplicates that increase the amount of DNA allowing for sequencing. These duplicates can be used to remove sequencing errors when two or more molecules with the same UMI are mapped back.

In an exemplary experiment, six PCR cycles were carried out using indexing primers for input masses above 5 ng, and 8 cycles were performed for <5 ng. Libraries were quantified as described above. To enhance duplicate recovery in human samples, 4 ng of prepared libraries was subjected to 6 additional PCR cycles prior to Ultima library conversion. Mouse PDX samples did not undergo additional PCR cycles prior to Ultima library conversion.

In step 106, the method includes selecting a subset of the whole genome library and amplifying the subset with a second PCR to increase an amount of PCR duplicates. As with the first PCR amplification, these duplicates can be used to remove sequencing errors when two or more molecules with the same UMI are mapped back.

In step 108, the method may include sequencing a series of reads from the amplified subset.

Illumina sequencing: Illumina sequencing libraries were sequenced on a HiSeq X or NovaSeq1.0 using 2×150 paired-end sequencing.

Ultima sequencing: Illumina sequencing libraries were sent to Ultima Genomics (Newark, CA) for library conversion and sequencing.

In step 110, the method includes aligning the reads to a host genome (e.g., the human genome) and denoising the duplex reads, using original sequencing reads and collapsed read information.

FastQ reads were adapter and UMI trimmed using cutadapt (version X). Trimmed reads were then aligned to the human genome (version hg38) using bwa mem (with parameters-K 100000000-p-v3-t 16-Y). Trimmed UMI's were added to the alignment files as an additional RX tag. Single-strand and duplex correction was carried out using the fgbio suite of tools (version 2.0). For single-strand error correction, reads were grouped by UMI (fgbio GroupReadsByU mi-s edit) and consensus calls were performed (fgbio CallMolecularConsensusReads—min-reads 2). Resulting error-collapsed fastQs were realigned to the human genome using bwa mem. For duplex error correction, single-strand consensus sequencing was performed independently on top-strand-mapping and bottom-strand-mapping reads. Bottom-strand mapping reads were subsequently reverse complemented and merged with top-strand mapping reads. Reads were then re-grouped by UMI and error correction was performed to obtain duplex corrected reads. Uncorrected reads that belonged to a duplex family were processed to measure the following read-specific features:
1. Base pair at the variant position
2. Edit distance of the read to the reference
3. Total number of single-nucleotide variants on the read
4. Read mapping quality
5. The position along the read from the extremities of the DNA fragment

In step 112 the method includes extracting a variant from the duplex reads. The type of variant is defined by a mutation, for example, a C base mutated into T (represented as C>T) and the base pairs adjacent to the mutated base. For example: A[A>T]A is one type of variant. A[A>C]A is another type of variant, as is A[A>G]A, A[C>A]A, etc. There are 96 types of variant.

Reads are filtered based on whether 1) all reads in a duplex family carried the same variant; 2) the edit distance was lower than 2; 3) the total number of single-nucleotide variants on the read was below 10; 4) the mapping quality was the highest possible value (60 for bwa mem); 5) the position along the read was greater or equal to 10.

In step 114, the method includes comparing the extracted variant to a collection of cancer specific variant signatures. Certain cancers have very well-defined variant types, i.e., cancer specific mutational signatures. Melanoma, for example, is a cancer with a distinct signature that is related to the skin's exposure to UV rays. Some lung cancers will show a signature associated with exposure to tobacco. This signature matching process is shown by FIG. 3E. The fraction measurement estimation represented along the y-axis is found by using copy-number based tumor fraction estimation.

Copy number analysis was performed using ichorCNA (version). Tumor fractions were estimated after correcting for library and sequencing artifacts via a panel of normals from cancer-free controls (CTRL-01 to CTRL-05) sequenced on the same instrument as the sample.

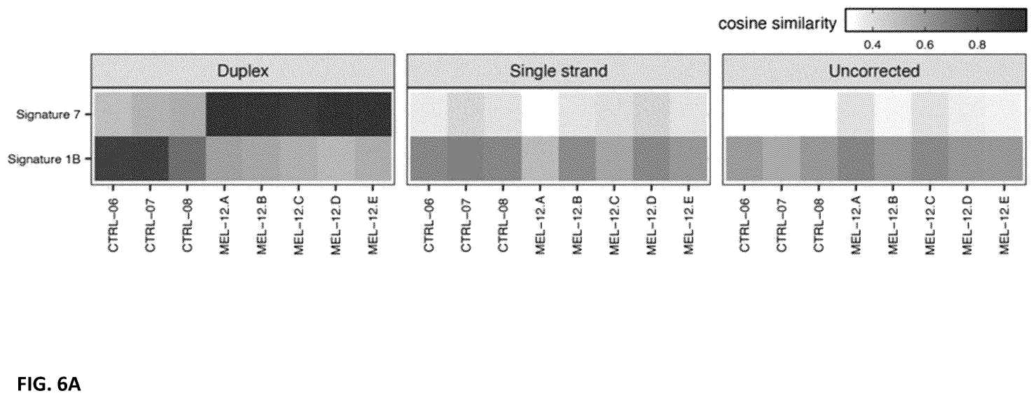

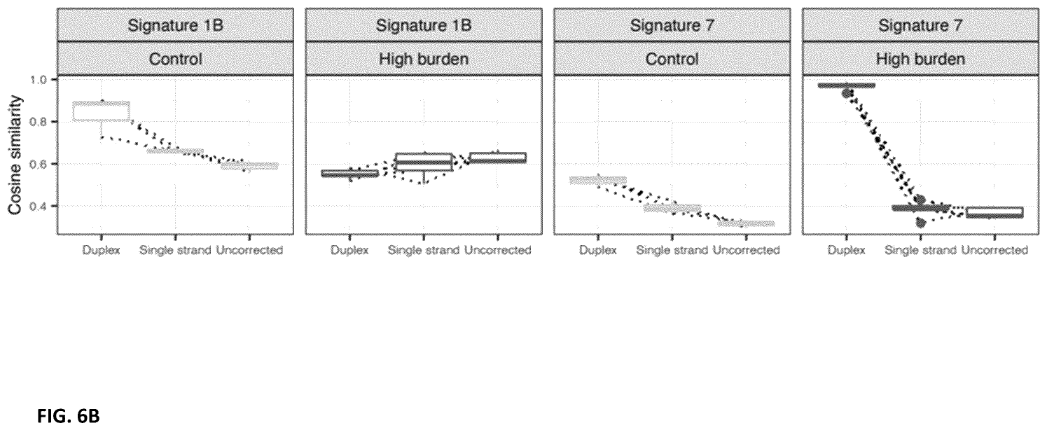

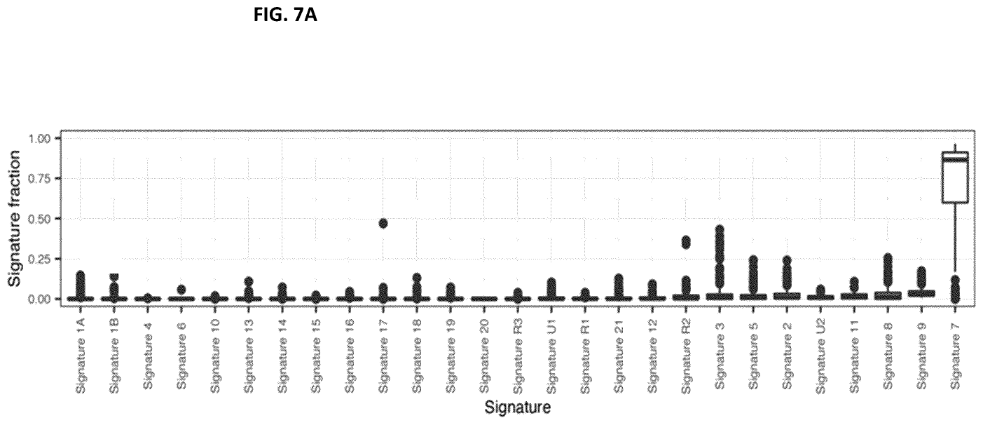

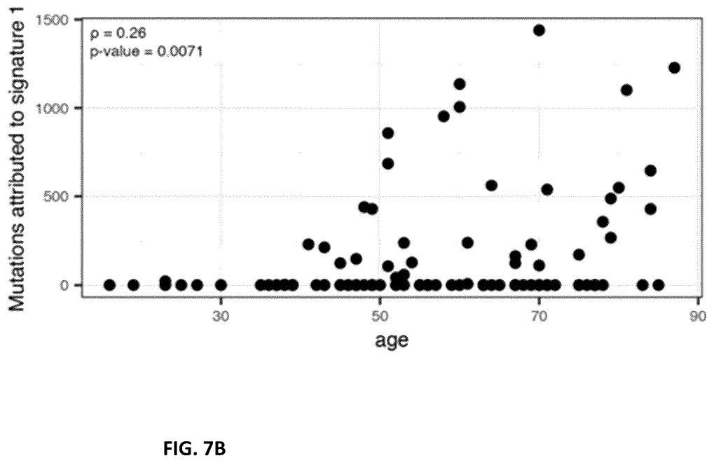

In low tumor burden settings, not all somatic mutations from the tumor are represented in the cell-free DNA sequencing pool, and any genomic locus is expected to be covered by at most one circulating tumor DNA read. Therefore, read-based TF estimation frameworks, and not locus-based TF estimations, are necessary to accurately quantify ctDNA content. Genome-wide mutations from the sequencing reads may be integrated and summarized as a weighted sum of single-base substitution (SBS) reference mutational signatures. Expectedly, in a re-analysis of publicly available PCAWG melanoma datasets, it was found that the UV-associated SBS7 mutational signature was most abundant in melanoma WGS datasets, and that clock-like signatures 1A/B weakly correlated with age of patients at tissue collection (spearman's ρ=0.26, p-value=0.0071, FIGS. 6A-6B). The trinucleotide contexts of cfDNA variants were explored through mutation signature analysis at each level of denoising (UMI-agnostic, single-stranded and duplex), in order to investigate the potential sources conferring mutations in these samples. Cosine similarities between the UV-associated SBS7 signature and high burden samples were highest after duplex correction (mean cosine similarities to SBS7 of 0.967±0.02 and 0.365±0.03 between duplex corrected and uncorrected samples, p-value=1.6×10⁻⁴ (Wilcoxon rank sum test)), FIGS. 7A-7B). Similar improvements were found when measuring cosine similarities between clock-like signatures and cancer-free controls, highlighting the importance of duplex correction for accurate signature analysis (FIGS. 7A-7B).

Given the ability of de novo mutation identification in error corrected cfDNA WGS to deliver profiles matching SBS7 and clock-like signatures for identifying melanoma and age-associated circulating DNA fragments, respectively, a tumor-agnostic approach for ctDNA detection was developed based on mutational patterns. As a first step, SBS mutational signatures are deconvolved from plasma ctDNA mixtures using a non-negative maximum likelihood model, and a tumor fraction is estimated by taking the weight of the tumor-associated SBS signature and normalizing by total number of mutations and depth of sequencing. Second, a signature score is calculated in order to determine whether the cancer-associated SBS signatures better explain the observed mutation profiles compared to a random permutation of the cancer-associated motifs. To analytically validate this approach, an in silico mixing study was conducted, combining duplex-denoised reads from two high burden ctDNA samples (MEL-12.A and MEL-12.B) and a cancer-free control (CTRL-06) at 10× sequencing depth (after duplex consensus), in varying proportions (expected tumor fractions from 0 to 1%). As a result, estimated tumor fractions were readily detectable at expected tumor fractions of 10⁻⁴ (receiving operating characteristic area under the curve 0.90), with signature scores highly specific for melanoma at 10⁻³ dilutions (FIG. 3F).

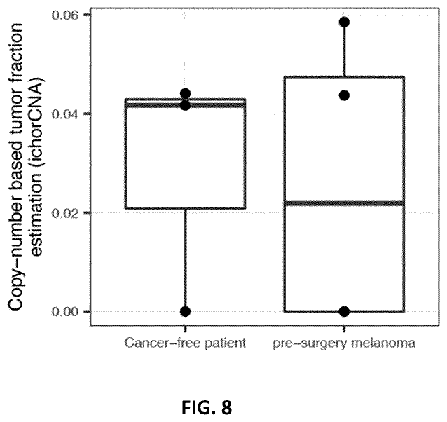

In an embodiment, a signature-based ctDNA detection platform for pre-operative ctDNA detection (i.e. tumor-agnostic ctDNA detection) was applied. Plasma samples were sequenced from four patients with stage III melanoma, three cancer-free controls, and one treatment-unresponsive patient (5 separate time points) with stage IV melanoma. Notably, signature scores for ctDNA detection showed perfect separation between cancer-free controls and samples from melanoma patients (FIGS. 3G-3H), while copy-number based analysis could not resolve these two groups (FIG. 8).

Tumor genotyping can be performed via cfDNA & normal tissue sequencing when the tumor burden in the plasma is high (>10%, sources). Mutect2 can be used with the normal tissue. A quality threshold was established, and only SNVs are kept. Then, four blacklists are applied to create a final tumor panel. (encode, gnomad, local blacklist, centromeres).

Read counting was performed in 1M bp bins using hmmcopy (excluding duplicates and reads with a mapping quality below 60). Read counts were adjusted for mappability and GC content using hmmcopy. Separate panels of normals were created for Illumina and Ultima datasets, respectively, using cancer-free controls (CTRL-01 to CTRL-05; n=5 per sequencing instrument). Tumor fraction estimates were obtained using ichorCNA (version). For plotting purposes (FIG. 1X, Supplemental Figure Y), corrected log 2 read counts outputted by ichorCNA were used. Bins marked by ichorCNA as copy gains, amplifications and high-level amplifications were marked and colored as chromosome gains (pink). Bins marked as homozygous deletion states and hemizygous deletions were marked and colored as chromosome losses (blue). Copy neutral regions were marked as neutral (black). Bins with corrected log 2 read counts between −0.05 and 0.05 were marked as neutral (black) as well.

In step 116, the method includes determining a cancer status based on a match between variant signatures.

Variants detected using the denoising method described above in were used. Variants with allele frequencies greater than 30% were presumed to be germline mutations and were discarded. Remaining reads were aggregated, and the frequencies of the variants in their trinucleotide context were calculated. These trinucleotide variant frequencies were compared to the trinucleotide variant frequencies of publicly available references for different biological processes. In this context, given that processed samples were from cancer-free controls and melanoma patients, it was assumed that the sample's trinucleotide variant frequencies would be a combination of aging-related trinucleotide frequencies and UV-damage associated trinucleotide frequencies. The sample's frequencies were fit to the references using a non-negative maximum likelihood method. To remove false positives, a permutation test was performed. This test involved randomly changing the trinucleotide frequencies of the UV-associated (melanoma) reference signature and performing the non-negative maximum likelihood fit. If the sample showed a stronger fit to the randomly-permuted frequencies than to the original one, it was deemed to be a false positive. This exercise was repeated 10,000 times to obtain a signature score. If the sample had a signature score below 0.001, it was deemed acceptable. Samples above this threshold were deemed cancer-negative.

In some embodiments, the method may include exhaustive WGS. In addition to WGS, embodiments of the present disclosure may use less exhaustive sequencing methods such as whole exome sequencing (WES) or SNP genotyping. Various enrichment modalities may be employed, including but not limited to: exome enrichment, targeted gene enrichment, and/or specific mutation enrichment. For example, exome enrichment may comprise whole exome sequencing. Targeted gene enrichment may include sequencing entire genes, including one or more of introns, exons, and/or coding sequences. A specific mutation enrichment may target a specific position of the genome, including, e.g., an exon, an intron, and/or some other user-defined position. Technologies to accomplish enrichment of at least one of the aforementioned regions are typically hybridization based or primer based. Such examples include: targeted variant sequencing; targeted gene sequencing; whole exome sequencing; targeted PCR; nested PCR; and/or linear PCR.

In cases where a limited input is used, such as in cell-free DNA, it is possible to exhaustively sequence the sample (i.e., sequence every single molecule available). Accordingly, some embodiments of the present disclosure may include exhaustive whole exome sequencing, exhaustive cfDNA sequencing, and/or exhaustive targeted sequencing.

FIGS. 2A-2F depict ultralow ctDNA detection requiring deep sequencing coverage and low error rates.

FIG. 2A is a collection of graphs showing a simulated sequencing coverage. Simulation analysis shows that lower error rates and high sequencing coverage are required for accurate ctDNA detection when tumor fractions are at or below 10⁻⁵.

Simulations for FIG. 2A were performed assuming a tumor-mutational compendium of 10,000 SNVs at different error rates (10-3, 10-4 and 10-5), coverages (1, 10 and 100) and tumor fractions (0, 10-6, 10-5). For each of the 50,000 SNV mutations, coverage was simulated using a poisson distribution. Each simulated sequenced base pair was classified as either ctDNA or cfDNA according to the tumor fraction, and errors misclassified as ctDNA were determined according to the error rate. Estimated tumor fractions were calculated by summing the ctDNA molecules and the errors, and dividing by the total base pairs simulated.

FIG. 2B is a pre-analytical workflow for cfDNA library preparation. The workflow, similar to that of the embodiment shown in FIG. 1, comprises obtaining plasma, from which cfDNA is extracted. The double stranded DNA library is prepared for sequencing, which is done using the Illumina sequencing method or Ultima library conversion and subsequent Ultima sequencing.

FIG. 2C is a graph comparing sequencing depth (genome equivalents) of matched Illumina and Ultima datasets, across 15 matched cfDNA samples.

FIG. 2D is a comparison of normalized read coverage for Illumina (top) and Ultima (bottom) matched cfDNA samples (chromosomes).

FIG. 2E is a comparison of copy number variations (CNV) tumor fraction and single-nucleotide variants (SNV) tumor fraction using Illumina and Ultima datasets. On the left graph, the CNV tumor fraction estimation measured with Illumina or Ultima sequencing is shown in matched samples using ichorCNA. Matched cancer-free controls were used to create a panel of normal prior to tumor fraction estimation.

On the right graph of FIG. 2E, single nucleotide variant-based tumor fraction estimation measured with Illumina or Ultima sequencing is shown. Somatic SNVs were identified through matched tumor-normal sequencing. Two samples without tumor sequencing and with low ctDNA fraction (e.g., less than 5% measured through CNV analysis) were omitted.

FIG. 2F depicts an expected tumor fraction score with and without error suppression. An in silico mixing study of metastatic melanoma sample MEL-01 with cancer-free control CTRL-05 (50 replicates per tumor fraction, 80× coverage per replicate) show the effect with (red) and without (blue) tumor-informed analytic denoising applied using Ultima-specific quality filtering.

FIGS. 3A-3H depict duplex correction allowing ctDNA without tumor sequencing. FIG. 3A depicts error rates in mouse and human DNA among duplex sequencing, single strand sequencing, and uncorrected groups. The graph on the left shows error rates for duplex WGS sequencing on mouse PDX samples (n=3). Open circles in the graph on the left represent samples for which no sequencing errors were detected. The graph on the right represents duplex WGS sequencing in patient sample MEL-12.D intersected with tumor mutation profiles of 107 melanoma patients retrieved from the Pan Cancer Analysis of Whole Genome Consortium. Base changes matching the somatic mutation of the tumor were considered errors (after removing germline and somatic mutations from the matched patient data).

To first test the accuracy of duplex error correction, duplex libraries were prepared using cfDNA obtained from the plasma of mice with patient-derived xenografts (n=4, NOD/ShiL tj species; n=1 lung cancer; n=3 diffuse large B cell lymphoma). Tumor fractions, defined as the fraction of reads uniquely mapping to the human genome, were 0.4%, 40%, 73% and 96%. To estimate the error rate of the duplex libraries, mutation levels were investigated at well characterized homozygous variant sites for NOD/ShiL tJ mice in the three samples with elevated mouse-mapped reads. Overall, only two bases out of over 4.2×10⁶ total bases were sequenced that were inconsistent with the known genotype of the mice for an error rate of 4.75×10⁻⁷ (FIG. 3A). These results are consistent with a previous report employing whole genome duplex sequencing (A bascal et al, 2021 reports error rates of 2×10⁻⁷ using similar protocols).

FIG. 3B is a series of graphs comparing variant allele frequencies calculated using unfiltered sequencing reads. Variant allele frequencies are shown in positions where a variant was found using uncorrected reads (left column) and in duplex corrected reads (right column). Top and bottom rows are representative examples for cancer-free and high-burden patient samples, respectively.

FIG. 3C is a graph comparing the model allele fraction of a patient with progressive disease (samples MEL-12.A-E) in duplex corrected positions (allele fractions below 30% only) and copy-number based tumor fraction estimations.

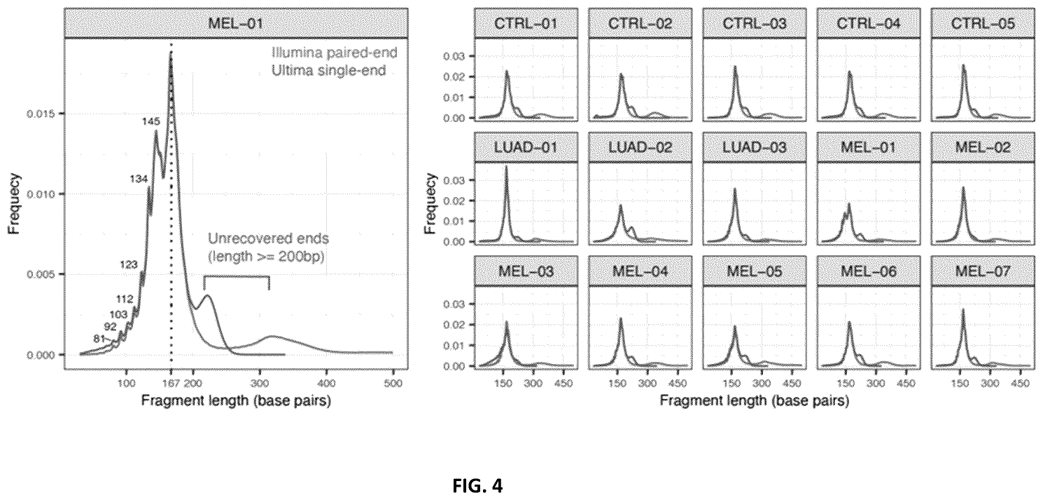

FIGS. 3D and 3E illustrate an exemplary method of signature matching between sequencing reads. The signature 7 reference of FIG. 3D is a publicly available signature associated with UV exposure (i.e., a melanoma specific signature). The signatures of FIG. 4E show uncorrected, single-strand correction, and duplex correction of a control signature and a melanoma patient with a MEL-12 D signature. Each bar on the signature represents a specific trinucleotide mutation (i.e., there are 96 bars) and the y-axis shows the relative proportion of trinucleotide mutation. The duplex corrected MEL-12 D signature is matched to the reference signature, and the cancer-like signature is only apparent in the cancer patient after duplex correction.

FIG. 3F is a graph illustrating a signature score and ctDNA detection of an in silico mixing study of metastatic melanoma samples MEL-12.A/B with cancer-free control CTRL-06 (10 replicates per tumor fraction, 10× coverage per replicate).

In the top row, the signature score is used to estimate the contribution of signature SBS& (melanoma UV associated) in the decomposition of a sample's trinucleotide frequencies into reference signatures.

In the bottom row, ctDNA detection by expected tumor fraction. Z-scores estimation was used to calculate mutation signature SBS7 detection in comparison to detection in TF=0 replicates. Ground truth variants originating from either the high-burden sample MEL-12.A/B. or the cancer-free sample CTRL-06 are shown in blue (full circle: MEL-12.A/B; open circle: CTRL-06). Error bars represent the standard deviation in the number of variants per replicate at a given expected tumor fraction.

FIG. 3G is a graph illustrating a series of signature scores of melanoma signature 7 in plasma cfDNA samples using duplex WGS (n=9 melanoma samples; n=3 controls). Samples in red are from patient MEL-12 with stage IV melanoma at different time points in their clinical course. Samples in blue each represent a separate patient (MEL-08 to MEL-11). Samples in pink represent control samples.

FIG. 3H is a graph illustrating estimated tumor fraction of samples with elevated signature scores. The X-axis depicts the clinical timepoint for each patient sample. Tumor fractions were estimated by multiplying the number of single nucleotide variants found in duplex corrected reads by the weight of signature 7 after reference signature decomposition and normalization by depth of coverage. An assumed 10,000 SNV tumor mutational profile was assumed for tumor fraction estimation.

FIG. 4 depicts cfDNA fragment lengths in single-end sequencing datasets matched with paired-end sequencing. Fragment lengths are accurately recovered between single-end Ultima reads when compared to paired-end Illumina sequencing for cfDNA molecules below 200 base pairs.

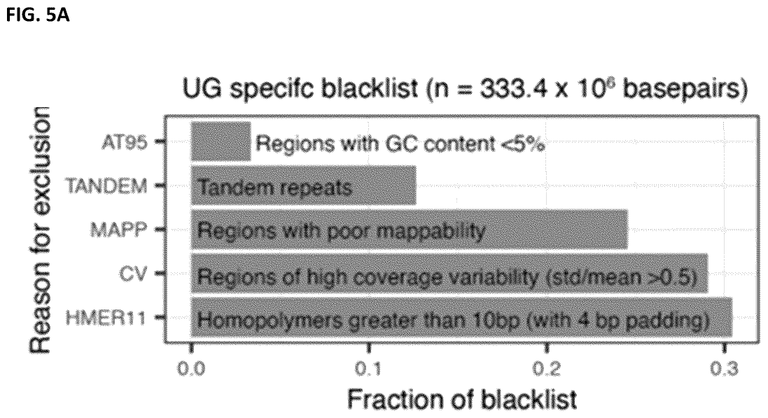

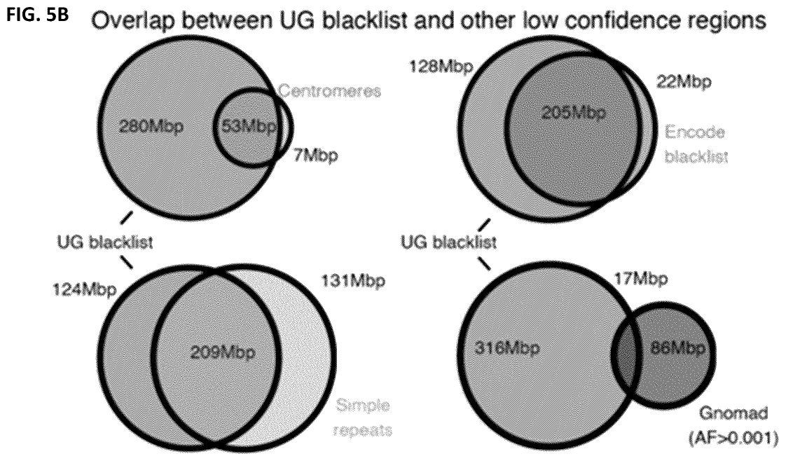

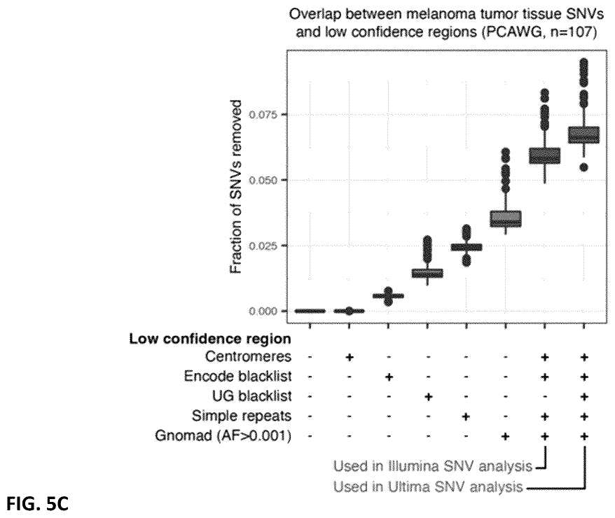

FIGS. 5A-5C depict the effective of artifact blacklisting on a single nucleotide variant detection.

FIG. 5A is a graph illustrating the UG specific blacklist. The UG specific blacklist includes regions with low GC content, tandem repeats, regions with poor mappability, regions with high coverage variability and regions with homopolymers greater than 10 base pairs.

FIG. 5B is an illustration of overlap between the UG blacklist and other low confidence regions. Other low confidence regions include centromeres, simple repeats, regions that encode blacklist, and gnomad regions with A F value greater than 0.001.

FIG. 5C is a graph illustrating the overlap between melanoma tumor tissue SNVs and low confidence regions. The effects of blacklists on the recovery of somatic single nucleotide variants (SNVs) are shown in 107 melanoma tissue samples obtained from the Pan Cancer Analysis of Whole Genomes consortium.

FIGS. 6A-6B depict cosine similarities in high burden and cancer-free samples for clock-like and UV-associated signatures SBS1B and SBS7, respectively.

FIG. 6A is a heatmap of cosine similarities in cancer-free samples and high-burden ctDNA samples. The heatmap of cosine similarities in duplex-corrected, single strand corrected and uncorrected reads from cancer-free samples (n=3) or high burden ctDNA samples (n=5, all from patient MEL-12 with stage IV B melanoma). The x-axis is ordered alpha-numerically, without hierarchical clustering.

FIG. 6B is a boxplot of cosine similarities for three correction methods in the same cancer-free samples and high-burden ctDNA samples, according to techniques disclosed herein.

FIGS. 7A-7B depict the re-analysis of 107 melanoma mutational signatures from the Pan-Cancer Analysis of Whole Genomes consortium.

FIG. 7A is a graph showing the signature fraction of a number of variant signatures. Deconvolution of duplex-corrected mutations into representative mutational signatures was performed using a non-negative maximum likelihood model. Boxplots are ordered in increasing order of the median for each signature.

FIG. 7B is a correlation plot between age at cancer diagnosis and the number of clock-like mutations attributed to SBS1A and SBS1B, The number of mutations was obtained by multiplying the weights of SBS1A and SBS1B by the total number of mutations found after duplex correction.

FIG. 8 depicts tumor-agnostic copy-number based tumor fraction estimation in cancer-free control samples (n=3) and pre-surgery melanoma plasma (n=4).

In another embodiment, whole genome sequencing may occur without duplex, to reach an SNV-based tumor fraction estimation.

SNV-based tumor fraction estimation was carried out by counting cell-free DNA reads with matching tumor-specific somatic mutations (mutation calling pipeline described below). To limit the effect of problematic regions of the genome, a platform-specific blacklist was built. For Illumina sequencing, regions identified in the ENCODE blacklist (source), centromeres (source), simple repeat regions (source) and positions with high mutation rates (GNOMAD, AF>0.001, source) were not considered. For Ultima sequencing, Ultima-specific low-confidence regions composed of homopolymers, AT-rich regions, tandem repeats and regions with poor mappability and high coverage variability were additionally excluded.

To limit the effect of sequencing errors, custom scripts were used for platform-specific denoising. Illumina alignment files were filtered to contain read pairs overlapping somatic mutation positions. Paired-end reads were filtered for X, Y, Z and were only kept if both R1 and R2 carried the somatic mutation or the reference base pair. Tumor fractions were estimated by dividing the number of filtered reads containing the somatic mutation by the total number of filtered reads.

Ultima alignment files were subset to reads overlapping with somatic mutation positions. Reads were filtered by X, Y, Z. Tumor fractions were estimated by dividing the number of filtered reads containing the somatic mutation by the total number of filtered reads.

### SNV Model Training Sets and Feature Space

Training sets were obtained from plasma enriched for ctDNA SNV fragments (true label) from specific melanoma tumors and cfDNA SNV reads (false label) from healthy controls without known cancer as listed in sup tab xx. Candidate reads were extracted from custom denoised alignment files. For true label sets, patients with high burden metastatic disease were used and only reads which represented matched tumor variants were retained.

A custom deep-learning model is used for signal to noise enhancement, similar to previous work (Widman et al, 2022), and effectively categorized candidate SNV reads. Candidate SNV reads were extracted using pysam (v0.15.2). Additionally, compelling regional and sequencing tech specific features were encoded as input to the deep learning model architecture with a custom python (v3.6.8) script. Two separate input structures are described below, corresponding to each component of the ensemble model.

For the MLP, a tabular set of feature values is provided as an input.

The feature selection for this was performed on SNV reads post filtering in both the true and false label settings. Specific features and their corresponding single variable AUC performance is described in sup tab xx. As highlighted in previous work (Widman et al, 2022), tissue-specific transcriptional features aid in defining the likelihood for observing somatic mutations in a genomic region. Local tumor mutation density is categorized by quantifying WGS SNV mutation calls from the PCAWG database (edge ref 81) and the total number of SNV mutations are counted from available melanoma derived tumor samples. Additionally, local histone CHIP-Seq marks and tissue specific bulk RNA expression values were reported as standard RPKM values from primary tissue alignments in ENCODE (edge ref 95). Regional DNase peaks (lifted to GR Ch38) were also included, which were obtained from narrowpeak files as reported in ENCODE (edge ref 95,96). Melanoma specific ATAC peak calls as reported in TCGA (edge ref 82) were also included.

Since the deep learning model is designed to operate on a read level compendium, values for the features defined above were computed using a sliding window around each candidate read. The optimal length for this sliding window was defined in previous work (Widman et al, 2022). Additionally, regional chromatin annotation tracks (ChromHMM-lifted to GRCh38) (edge ref 83) were obtained from ENCODE. Hi-C SNIPER (edge ref 97) bed files were used to extract HI-C compartment information. Lastly, regional features for replication timing and mean expression values (lifted to GRCh38) were pulled from previous literature (edge ref 37).

In addition, Ultima specific read level features were included. These include the following:
X-FC1-number of features (SNPs) on the same read
X-FC2-number of features (SNPs) on the same read that passed the filter (matching the reference for −+5 bases)
X-FLAGS-propagated from the bam file flag.
ed_1-edit (Levenshtein) distance of the read from the reference, before SNV of interest.
ed_2-edit (Levenshtein) distance of the read from the reference, after SNV of interest.

Next for the CNN a one-hot encoded tensor structure of the candidate read was used, similar to previous work (Widman et al, 2022). Each read is encoded with a variant (sequencing artifact/noise from healthy controls or somatic mutation from high burden tumor plasma sample). The encoded tensor has an image-like structure with a shape of 12×240. The rows correspond to one hot encoded nucleotides (N,A,C,T,G) corresponding to the reference and the read. The penultimate row dimension is used to mark the position along the read highlighting the SNV of interest. Lastly, the absence/presence (0/1) of a cycle skip (as defined by Ultima) is encoded along the last row dimension to add further relevance to tri-nucleotide context of the SNV of interest. The columns correspond to individual nucleotides along the length of the read. While reads have a maximum length of 200, the extra 40 base pairs are padded with the reference genome thereby adding additional relevant contextual information.

### SNV Model Design and Training

The deep-learning model has an ensemble structure and consists of two major components—a regional/read specific multi layer perceptron (MLP) and a sequence based convolutional neural network (CNN), whose weight matrices are jointly learnt.

The MLP which takes a feature matrix as input consists of a linear stack of four dense blocks. Each block is defined as consisting of a fully connected layer with a ReLU activation. Furthermore, for the purpose of regularization the input to each fully connected layer is batch normalized and the output is passed through a dropout layer.

The CNN consists of four one dimensional convolution layers with non-linear ReLU activations, which extract sequential information at different spatial resolutions. Moreover, as in classical deep learning frameworks, each convolution layer (post nonlinear activation) is followed by a max pooling layer. The output is then passed through a stack of 3 dense blocks as defined above.

Subsequently, the latent output of both the MLP and CNN is then concatenated and passed through a single dense block. Finally, a probability score between 0 (sequencing noise) and 1 (true somatic mutation) is obtained by using a single sigmoid-activated fully-connected layer. This probability score reflects the model's estimate on whether a candidate SNV mutation present in the encoded read is likely from signal or noise. The ensemble model is built in K eras (v.2.3.0) with a Tensorflow base (1.14.0).

To train the ensemble model, the objective function defined as a binary cross entropy loss is minimized. Performance metrics were reported within balanced sets.

### UMI Correction Improves Insertion-Deletion Mutation (Indel) Detection Accuracy in Ultima Sequencing Datasets

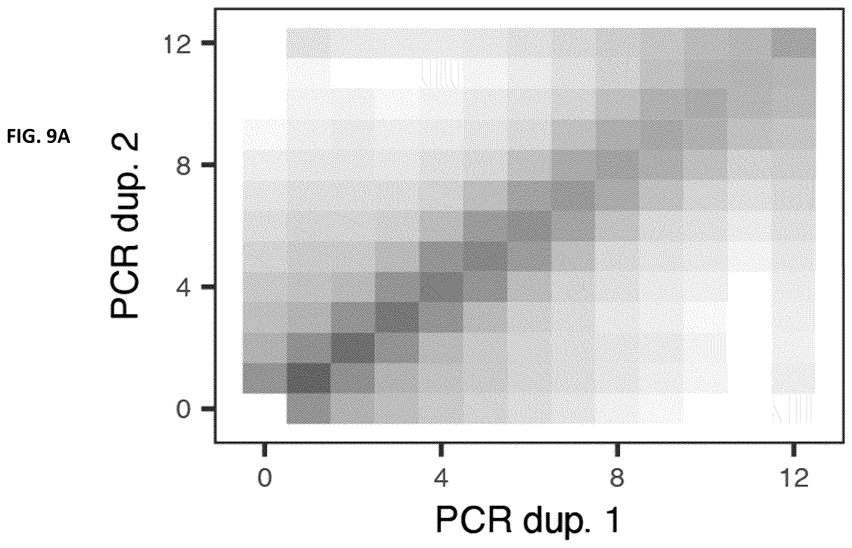

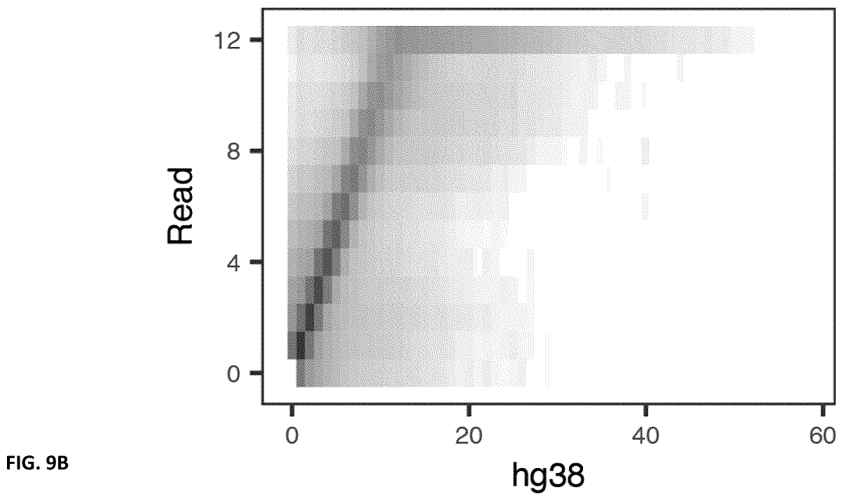

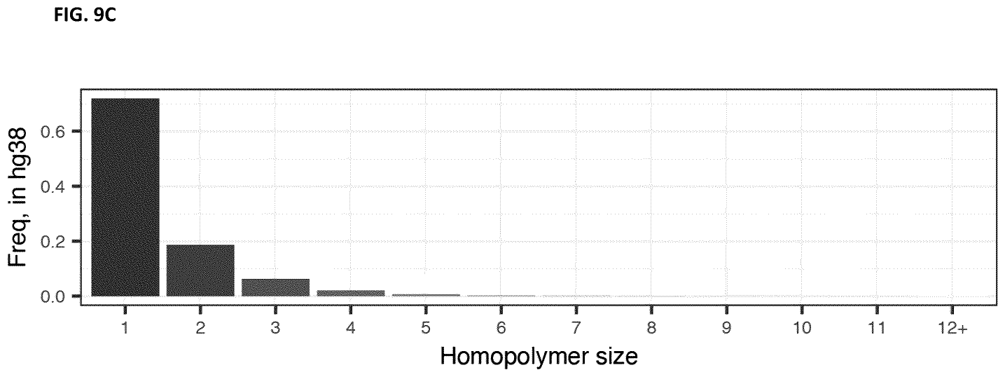

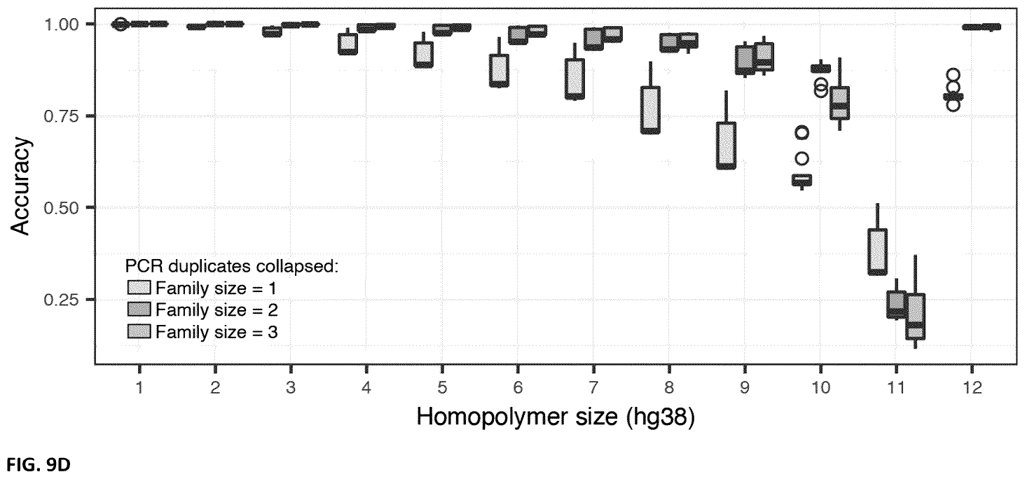

UMIs add a unique barcode to each DNA molecule. During PCR, the barcode tag (and DNA molecule) is duplicated multiple times. PCR duplicates can be thereby identified using the UMI. Identified duplicates can be used to correct sequencing errors, as it is unlikely that the same error will occur on two PCR duplicates. It should be noted that the Ultima flow-based sequencing is prone to homopolymer size errors, which are interpreted as false indel. FIG. 9A is a graph illustrating a homopolymer size between two PCR duplicates, whereas FIG. 9B is a graph illustrating a homopolymer size between a read and an aligned reference. For reference, FIG. 9C is a graph illustrating frequency of homopolymer size across the human genome, according to techniques disclosed herein. To further illustrate the increased accuracy of UMI correction, FIG. 9D is a graph illustrating insertion-deletion mutations indel calling accuracy by PCR duplicate family sizes, according to techniques disclosed herein.

### UMI Ligated Reads Allow for the Detection of Error Robust Trinucleotide Motifs in Ultima Sequencing Datasets

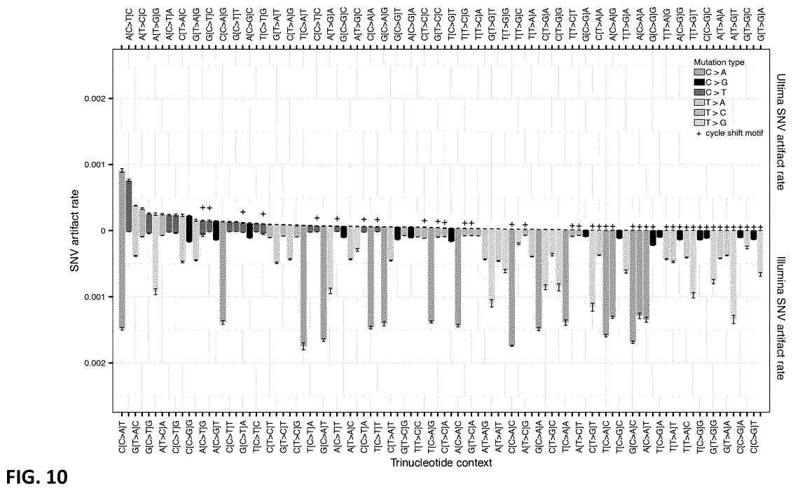

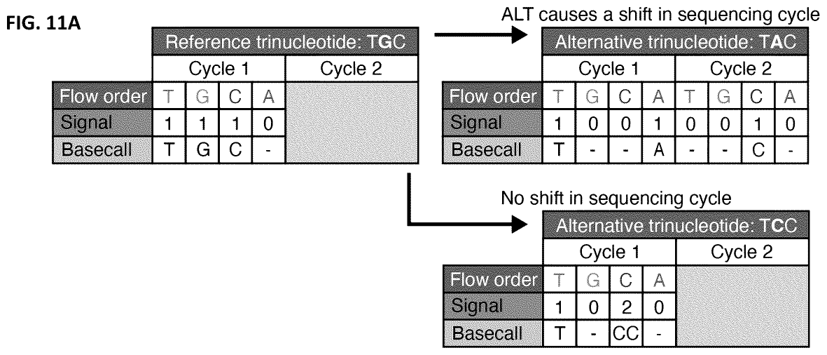

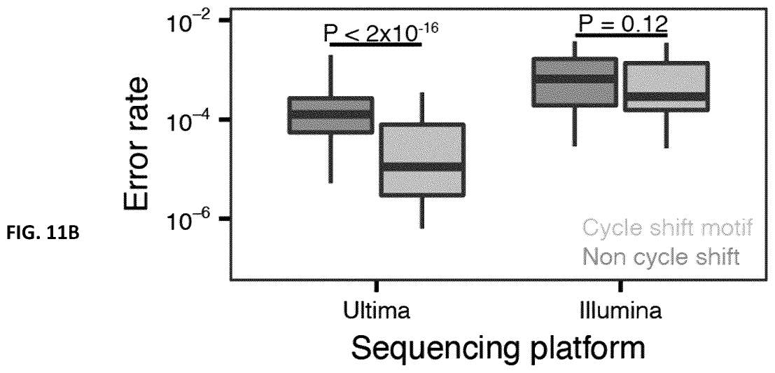

UMIs can also be used to find error-robust single nucleotide variants. These variants generally fall in cycle shift motifs, which are specific trinucleotides that Ultima has determined to be robust to errors. Details on cycle shifts are shown in FIG. 10, a graph illustrating a single nucleotide variant analysis of matched Ultima and Illumina sequencing datasets. FIGS. 11A-B show that flow-based sequencing provides predictable error-robust motifs. FIG. 11A is a flow chart of a sequencing process providing predictable, error-robust motifs, and FIG. 11B is a graph of error rate by sequencing platform, according to techniques disclosed herein.

### Wetlab Improvements and the Development of a Novel Machine Learning Classifier for Duplex Variant Detection

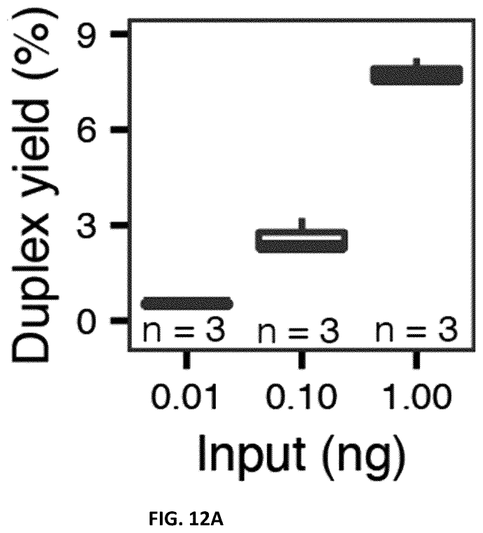

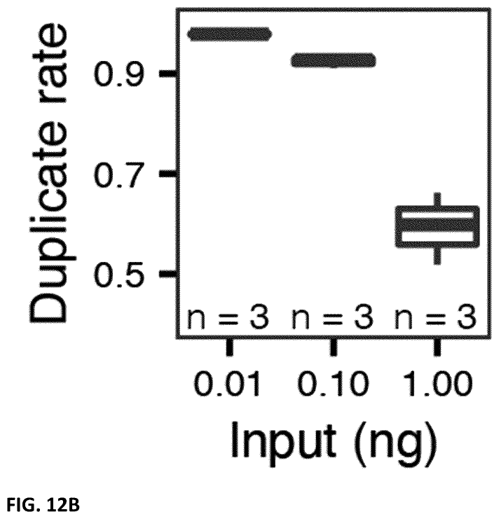

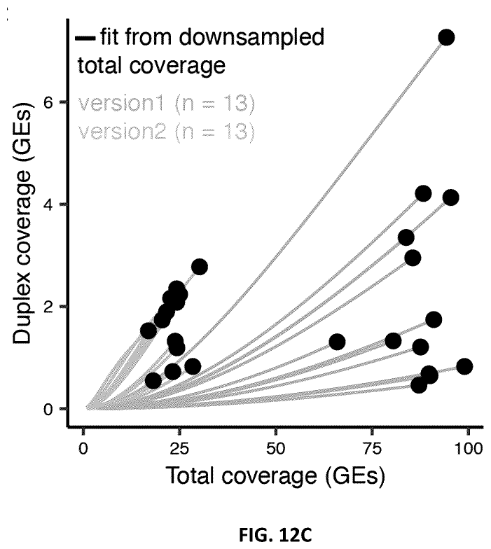

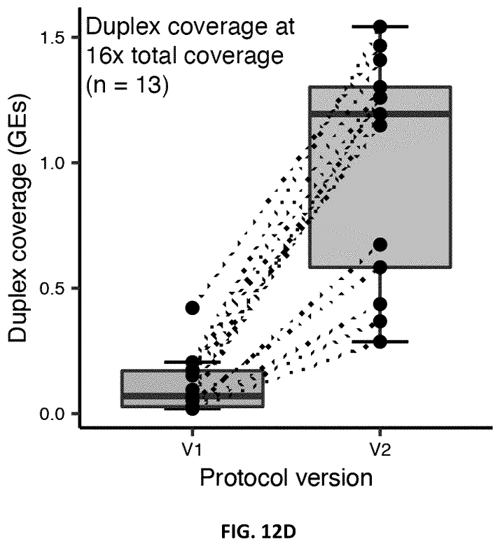

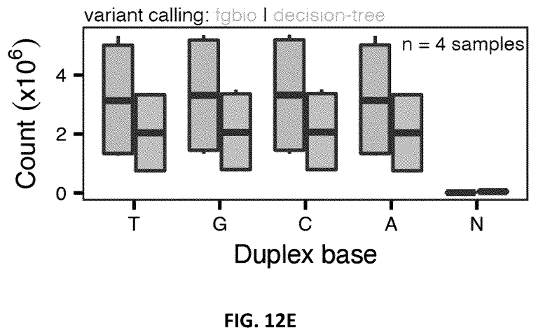

In some embodiments of the present disclosure, molecular and computational improvements were made to the methodology to improve yield of duplex molecules. The molecular improvement is shown by a more efficient bottlenecking of DNA molecules (FIG. 12A-B). FIG. 12A is a graph of duplex WGS libraries from three starting inputs sequenced at 1-13× coverage. Duplex correction was applied, and the yield of duplex recovery (depth of duplex-only coverage by total sequenced coverage) was measured, as shown in the graph. FIG. 12B illustrates the duplication rate of the samples of FIG. 12A at n=3.

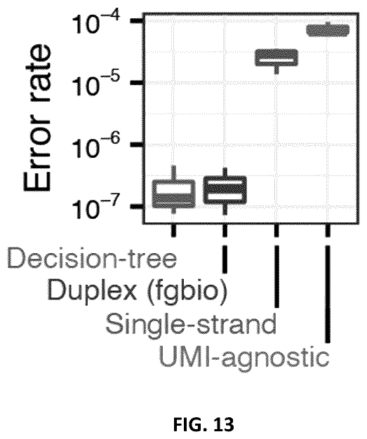

In FIG. 12C, a downsampling experiment shows that improved bottlenecking achieves higher duplex coverage at a faster rate than other embodiments. This is further illustrated by FIG. 12D, which is a a graph illustrating that duplex coverage is significantly higher at fixed coverage. FIG. 12E is a graph illustrating a number of duplex variants found using the duplex method (fgbio) versus a decision tree. FIG. 13 furthers this illustration with mutational error rates in mouse PDX samples (with N=3 per condition). This data illustrates the expanded applicability of the processes of the disclosed embodiments, including application to melanoma, stage III, with a baseline timepoint.

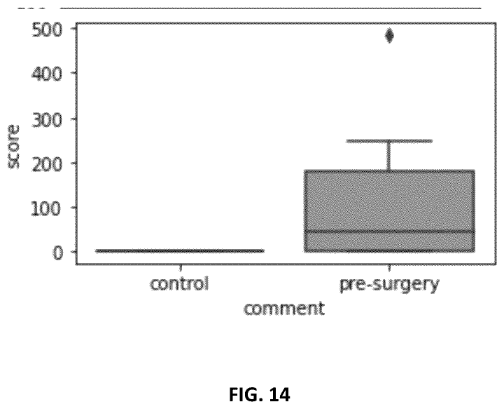

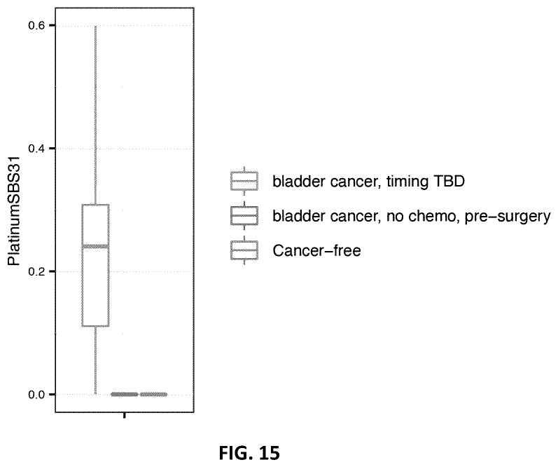

Some embodiments of the present disclosure produce more samples, including pre-surgery samples as shown in FIG. 14. Plasma cell-free DNA was obtained from patients with bladder cancer who may or may not have received chemotherapy. FIG. 15 shows that embodiments of the present disclosure detect a chemotherapy mutational signature in most samples that may have received chemotherapy, and specifically illustrates the application to bladder cancer, and the detection of an “APOBEC” signature and chemotherapy. In samples who never received chemo (green) or cancer-free controls (blue), the chemotherapy signal is not measured.

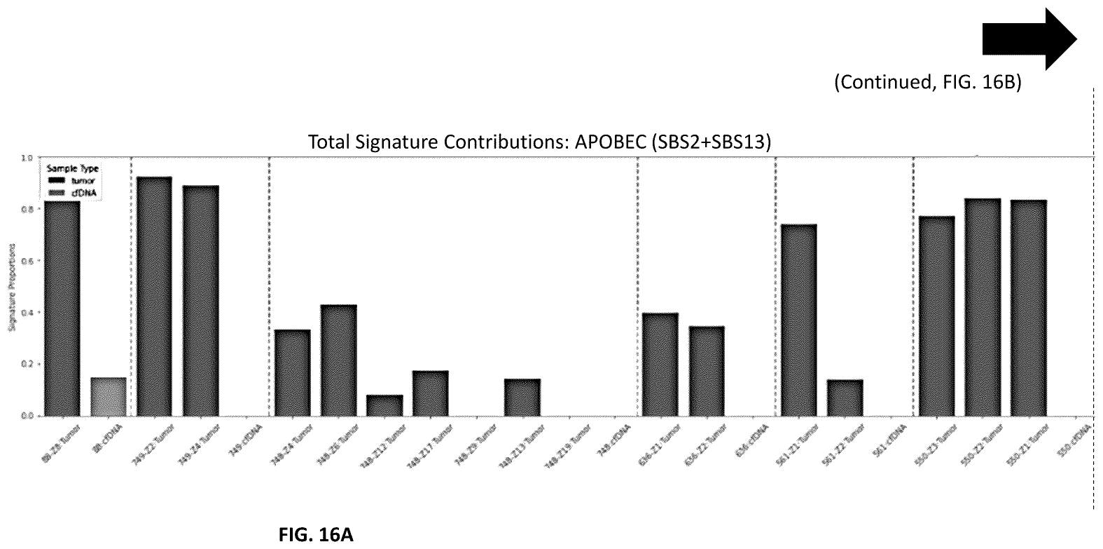

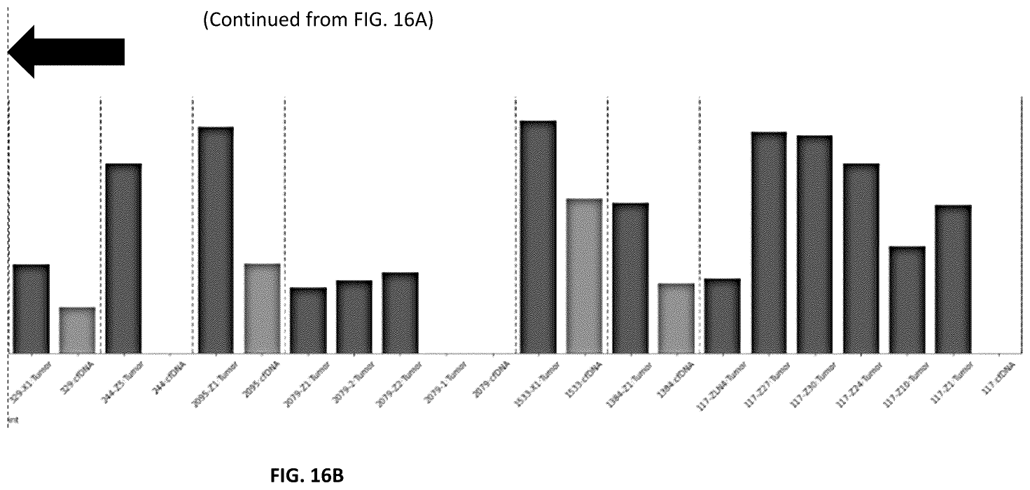

Bladder cancer typically shows the APOBEC mutational signature. This signature can also be detected in the plasma cell-free DNA, as shown in FIGS. 16A-B. Darker bars represent the APOBEC signature of tumors, and the lighter bars represent the APOBEC measurement in cfDNA.

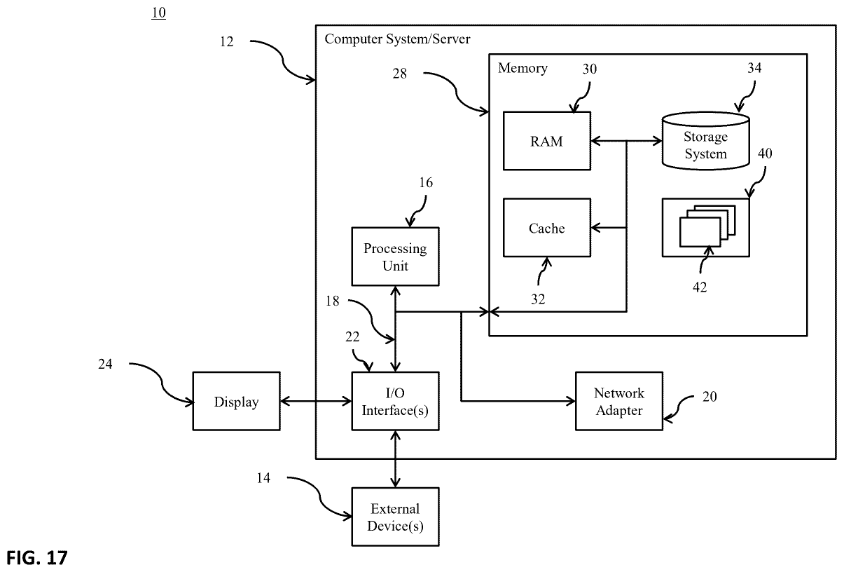

FIG. 17 is a schematic of an example of a computing node. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12 which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed computing environments where tasks are performed by remote processing devices that are linked through a communications network. In a distributed computing environment, program modules may be located in both local and remote computer system storage media including memory storage devices.

As shown in FIG. 17, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12 and it includes both volatile and non-volatile media, removable and non-removable media.

System memory 28 can include computer system readable media in the form of volatile memory, such as random access memory (RAM) 30 and/or cache memory 32 Algorithm Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42 may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12 and/or any devices (e.g., network card, modem, etc.) that enable computer system/server 12 to communicate with one or more other computing devices. Such communication can occur via Input/Output (I/O) interfaces 22 Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

In various embodiments, a learning system is provided. In some embodiments, a feature vector is provided to a learning system. Based on the input features, the learning system generates one or more outputs. In some embodiments, the output of the learning system is a feature vector. In some embodiments, the learning system comprises an SVM. In other embodiments, the learning system comprises an artificial neural network. In some embodiments, the learning system is pre-trained using training data. In some embodiments training data is retrospective data. In some embodiments, the retrospective data is stored in a data store. In some embodiments, the learning system may be additionally trained through manual curation of previously generated outputs.

In some embodiments, the learning system, is a trained classifier. In some embodiments, the trained classifier is a random decision forest. However, it will be appreciated that a variety of other classifiers are suitable for use according to the present disclosure, including linear classifiers, support vector machines (SVM), or neural networks such as recurrent neural networks (RNN).

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

We claim:

1. A method comprising:
extracting DNA from a collection of samples from an organism;
preparing a sequence library with duplex adapters, wherein the sequence library is prepared by ligating a duplex adapter having a Unique Molecule Identifier (UMI) to an end of each of a plurality of strands of the extracted DNA and amplifying the extracted DNA with a first PCR;
selecting a subset of the sequence library;
amplifying the subset with a second PCR to increase a number of PCR duplicates;
sequencing a plurality of duplex reads from the amplified subset;
aligning the plurality of duplex reads to a host genome and denoising the plurality of duplex reads based on said alignment;
detecting the presence of a variant in at least one of the plurality of duplex reads;
determining a signature of the variant;
comparing the signature of the variant to a collection of disease-specific variant signatures; and
determining a disease type based on the comparison.

2. The method of claim 1, wherein the UMI has exactly three base pairs.

3. The method of claim 1, wherein the UMI has less than five base pairs.

4. The method of claim 1, wherein the disease type is a cancer type.

5. The method of claim 1, wherein the cancer type comprises bladder cancer.

6. The method of claim 1, wherein the sequence library comprises one of a whole genome library or a whole exome library.

7. The method of claim 6, wherein preparing the sequence library with duplex adapters further comprises collapsing one or more errors on a strand of extracted DNA.

8. The method of claim 1, wherein amplifying the extracted DNA with the first PCR comprises removing sequencing errors based on the presence of two or more molecules with the same UMI.

9. The method of claim 1, wherein sequencing the plurality of duplex reads comprises sequencing on a paired-end system.

10. The method of claim 1, wherein
sequencing the plurality of duplex reads comprises sequencing on a single-end system and, wherein
aligning the plurality of duplex reads to the host genome comprises:
obtaining a plurality of single-end DNA sequencing reads;
separating a top-mapping strand of the DNA from a bottom-mapping strand of DNA;
performing error collapsing on each of the top-mapping strands and the bottom-mapping strands;
reverting the bottom-mapping strands to top-mapping strands by re-grouping based on UMI; and
performing error correcting between the top and bottom strands.

11. The method of claim 1, wherein
sequencing the plurality of duplex reads comprises sequencing on a single-end system and, wherein
aligning sequences to a host genome comprises:
obtaining a plurality of single-end DNA sequencing reads;
creating a synthetic paired-end read; and
performing error correcting on all strands.

12. The method of claim 1, wherein sequencing the plurality of duplex reads comprises sequencing a series of uncorrected reads belonging to a duplex family.

13. The method of claim 12, further comprising processing uncorrected reads belonging to a duplex family to measure a read specific feature.

14. The method of claim 13, further comprising filtering the uncorrected reads based on the measured read specific feature.

15. The method of claim 1, further comprising trimming the sequence of reads from the amplified subset.

16. The method of claim 1, wherein comparing the extracted variant to a collection of cancer-specific variant signatures comprises:
calculating a tumor fraction estimation of a duplex-corrected signature;
plotting the tumor fraction estimation to create a duplex-corrected signature for the extracted variant; and
matching the duplex-corrected signature to a reference signature.

17. The method of claim 16, wherein the signature comprises a relative proportion of a trinucleotide mutation.

18. The method of claim 4, further comprising:
correcting for library and sequencing artifacts by a panel of cancer-free controls sequenced on the same system; and
estimating a tumor fraction.

19. The method of claim 1, further comprising integrating a genome-wide mutation from the sequencing reads as a weighted sum of single-base substitution (SBS) reference mutational signatures.

20. The method of claim 19, wherein integrating the genome-wide mutation comprises:
deconvolving SBS mutational signatures from plasma DNA mixtures using a non-negative maximum likelihood model;
estimating a tumor fraction by taking a weight of a tumor-associated SBS signature and normalizing by a total number of mutations and depth of sequencing; and
calculating a signature score to determine that the cancer-associated SCS explains an observed mutation profile.

21. The method of claim 1, further comprising discarding a variant with an allele frequency greater than 30%.

22. The method of claim 21, further comprising:
aggregating reads having a variant with allele frequency less than 30%;
calculating a frequency of variants in the aggregated reads trinucleotide context; and
comparing the calculated trinucleotide variant frequency with a number of reference frequencies for different biological processes.

23. The method of claim 1, wherein determining a disease status comprises:
randomly changing a trinucleotide frequency of a reference signature from the collection;
performing a non-negative maximum likelihood fit between the randomly-permutated trinucleotide frequency and a frequency of the signature; and
scoring the fit below a disease-negative threshold.

24. The method of claim 1, wherein the DNA is genomic DNA.

25. The method of claim 1, wherein the DNA is cell-free DNA (cfDNA).

26. The method of claim 1, wherein detecting the presence of the variant comprises:
providing the plurality of duplex reads to a pretrained machine learning model; and
receiving therefrom an indication of a base variant irrespective of comparative sequence length.

27. The method of claim 26, wherein the pretrained machine learning model comprises an artificial neural network.

28. The method of claim 27, wherein the artificial neural network is one of a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

29. The method of claim 26, wherein the pretrained machine learning model comprises a trained classifier.

30. The method of claim 29, wherein the trained classifier is a random decision forest.

31. The method of claim 1, wherein the collection of samples comprises a collection of plasma samples.

32. The method of claim 1, wherein the sequence library comprises a whole genome sequence library.
